(19) United States
(12) Patent Application Publication
(10) Pub. No.: US 2012/0130421 A1
(43) Pub. Date: May 24, 2012

(54) RATCHETING STAPLE FOR SURGICAL APPLICATIONS

(75) Inventors: Zachary Hafez, Columbia, MO (US); William Verbarg, Columbia, MO (US); Nicole Patino, Columbia, MO (US); Brad Snow, Columbia, MO (US)

(73) Assignee: THE CURATORS OF THE UNIVERSITY OF MISSOURI, Columbia, MO (US)

(21) Appl. No.: 13/164,540

(22) Filed: Jun. 20, 2011

Related U.S. Application Data
(60) Provisional application No. 61/397,987, filed on Jun. 18, 2010.
(60) Provisional application No. 61/516,097, filed on Mar. 29, 2011.

(51) Int. Cl. A61B 17/064; B21G 7/02; A61B 17/068

(52) U.S. Cl. 606/220; 227/175.1; 59/77

(57) ABSTRACT

A surgical staple device which accommodates various tissue thicknesses by employing unique modified surfaces with increased friction is disclosed. The staple device includes a U-shaped staple body with modified surfaces on its legs, whereas the modified surfaces may be designed as ratchet teeth, barbs, hooks, grooves, and channels. The staple device further includes a footplate to receive and engage the legs.

### CROSS-REFERENCE TO RELATED APPLICATIONS

This is a non-provisional and claims benefit to U.S. Provisional Application No. 61/397,987 entitled “Ratcheting Staple” filed on Jun. 18, 2010, and U.S. Provisional Application No. 61/516,097 entitled “Surgical Staple” filed on Mar. 29, 2011, both of which are hereby incorporated by reference.

### FIELD

The present application relates to surgical devices and more particularly to a ratcheting staple, which can be used to securely join tissues and blood vessels.

### BACKGROUND

During a surgical procedure, a conventional surgical staple is typically used to secure tissue sections together. Conventional surgical staples generally have two equal-length legs joined by a cross section. The staples have a general “U-shape” configuration, where the two legs are driven through the tissue secures and then deformed by an anvil to curve back towards the secured tissue.

The length of the staple legs used in a procedure is often dictated by the amount of tissue to be secured. Therefore, conventional staples come in a variety of sizes that must be continually restocked at medical facilities. Inadequate staple length can cause severe complications if the staple fails to secure the tissue. Such complications often go undiscovered until the patient is out of surgery or away from the medical facility. It is very costly to continually maintain a supply of different staples. In addition, using an inadequate staple length can be very dangerous.

### SUMMARY

In one embodiment, a surgical staple device includes a staple body defining a first leg, a second leg, and a base having a first end and a second end. The base has a first leg extending from the first end and a second leg extending from the second end. The first leg has a first surface and the second leg has a second surface, wherein the first surface and the second surface are modified surfaces to prevent retrograde motion of the surgical staple device. A footplate is mechanically engaged to the modified surfaces. The footplate also has a first footplate end defining a first channel to receive the first leg and a second footplate end defining a second channel to receive the second leg.

In another embodiment, a surgical staple device includes a staple body defining a first leg, a second leg, and a base having a first end and a second end. The first leg extends from the first end and the second leg extends from the second end. The first leg has a first inner surface and a first outer surface and the second leg has a second inner surface and a second outer surface, wherein the first inner surface and the second inner surface face have modified surfaces to prevent retrograde motion of the surgical staple device. A footplate is mechanically engaged to the modified surfaces, the footplate having a first footplate end defining a first channel to receive the first leg and a second footplate end defining a second channel to receive the second leg.

In yet another embodiment, a surgical staple device includes a staple body defining a first leg, a second leg, and a base having a first end and a second end. The first leg extends from the first end and the second leg extends from the second end. The first leg has a first outer surface and a first outer surface and the second leg has a second outer surface and a second outer surface, wherein the first outer surface and the second outer surface face have modified surfaces to prevent retrograde motion of the surgical staple device. A footplate is mechanically engaged to the modified surfaces, the footplate having a first footplate end defining a first channel to receive the first leg and a second footplate end defining a second channel to receive the second leg.

In one embodiment, a surgical stapler device includes an elongated shaft having a proximal end and a distal end. A handle is mounted on the proximal end of the elongated shaft and a staple device forming mechanism is mounted on the distal end of the elongated shaft.

The staple device forming mechanism includes a plurality of staple devices having a staple body defining a first leg, a second leg, and a base having a first end and a second end. The base has a first leg extending from the first end and a second leg extending from the second end. The first leg has a first surface and the second leg has a second surface, wherein the first surface and the second surface are modified surfaces to prevent retrograde motion of the staple body.

The staple device forming mechanism further includes a footplate mechanism containing a plurality of footplates. Each of the plurality of footplates is configured to engage the modified surfaces of one of the plurality of staple devices. Each of the plurality of footplates has a first footplate end defining a first channel to receive the first leg and a second footplate end defining a second channel to receive the second leg.

In another embodiment, method for manufacturing a surgical staple device includes modifying a first surface at a first end of an elongated material to form a first modified surface having at least one of a first plurality of projections or a first plurality of recesses. A second surface is modified at a second end of the elongated material to form a second modified surface having at least one of a second plurality of projections or a second plurality of recesses.

An open-ended staple body is formed from the elongated material by bending the first end to form a first leg and bending the second end to form a second leg. A footplate defining a first channel at proximal end of the footplate and defining a second channel at a distal end of the footplate is provided. The first channel further defines a first pawl and the second channel further defines a second pawl.

The first channel is configured to slidably engage the first leg and the first pawl is configured to mechanically engage the first modified surface. The second channel is also configured to slidably engage the second leg and the second pawl is configured to mechanically engage the second modified surface.

In one embodiment, a method for using a surgical staple device includes providing a surgical stapler device including an elongated shaft further having a proximal end and a distal end, a handle mounted on the proximal end of the elongated shaft. The surgical stapler device also has a staple device forming mechanism mounted on the distal end of the elongated shaft and a footplate mechanism mounted on the distal end of the elongated shaft opposite the staple device forming mechanism. The staple device forming mechanism further including a plurality of staple devices having modified surfaces and the footplate mechanism including a plurality of footplates configured to mechanically engage the modified surfaces.

A first portion of tissue is positioned adjacent to a second portion of tissue and the distal end of the elongated shaft is positioned to enclose the first portion of tissue and the second portion of tissue between the staple device forming mechanism and the footplate mechanism. The handle is manipulated to compress the first portion of tissue and the second portion of tissue between the staple device forming mechanism and the footplate mechanism. At least one of the plurality of staple devices pierces the first portion of tissue and the second portion of tissue and at least one of the plurality of footplates mechanically engages the at least one of the plurality of staple devices compressing the first portion of tissue and the second portion of tissue there between.

Additional objectives, advantages, and novel features will be set forth in the description which follows or will become apparent to those skilled in the art upon examination of the drawings and detailed description which follows.

### BRIEF DESCRIPTION OF THE DRAWINGS

FIG. 1 is a perspective view showing one embodiment of a ratcheting staple and a footplate;

FIG. 2 is a perspective view showing the ratcheting staple engaged with the footplate;

FIG. 3 is a cross-sectional view of the ratcheting staple engaged with the footplate;

FIG. 4 is an elevated perspective view of the footplate;

FIG. 5 is a is a perspective cross-sectional view of the footplate;

FIG. 6 is a top-down view of another embodiment of the of the footplate;

FIG. 7 is a perspective view showing one embodiment of a ratcheting staple and a footplate;

FIG. 8 is a perspective view showing the ratcheting staple of FIG. 7 engaged with the footplate.

FIG. 9 is a perspective view showing one embodiment of a ratcheting staple and a footplate;

FIG. 10 is a perspective view showing the ratcheting staple of FIG. 9 engaged with the footplate;

FIG. 11 is a perspective view showing one embodiment of a ratcheting staple and a footplate;

FIG. 12 is a perspective view showing the ratcheting staple of FIG. 11 engaged with the footplate;

FIG. 13 is a side view showing one embodiment of a stapler device;

FIG. 14 is a side view showing a sequence of one embodiment of the ratcheting staple being deployed;

FIG. 15 is a side view showing one embodiment of a stapler device; and

FIG. 16 is a side view showing a sequence of one embodiment of the ratcheting staple being deployed.

Corresponding reference characters indicate corresponding elements among the view of the drawings. The headings used in the figures should not be interpreted to limit the scope of the claims.

### DETAILED DESCRIPTION

Aspects of the present disclosure include devices and methods for using a ratcheting staple. In particular, the ratcheting staple device includes a staple body having two or more legs surface modifications that mechanically engage with a footplate to prevent retrograde motion of the staple body once secured to tissue. The ratcheting staple can accommodate a variety tissues and tissue thickness.

The ratcheting staple device may be formed from any appropriate material having desirable rigidness and flexibility appropriate for the intended use. For example, the ratcheting staple device may be composed of metals, alloys, plastics, composites, polymers, among others. The ratcheting staple device may also be composed of degradable materials that will dissolve or be absorbed by surrounding tissue over time.

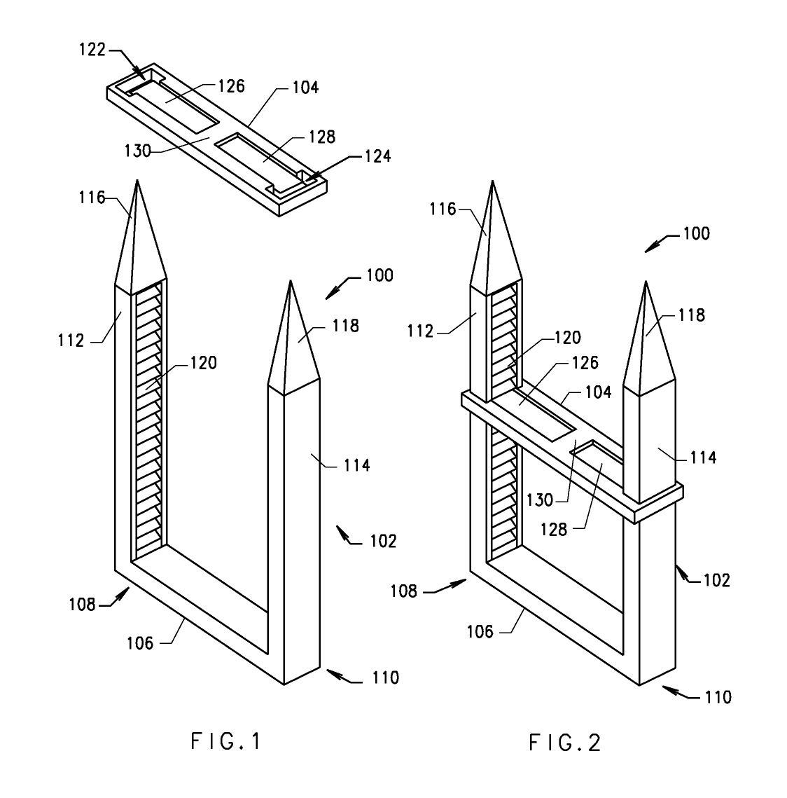

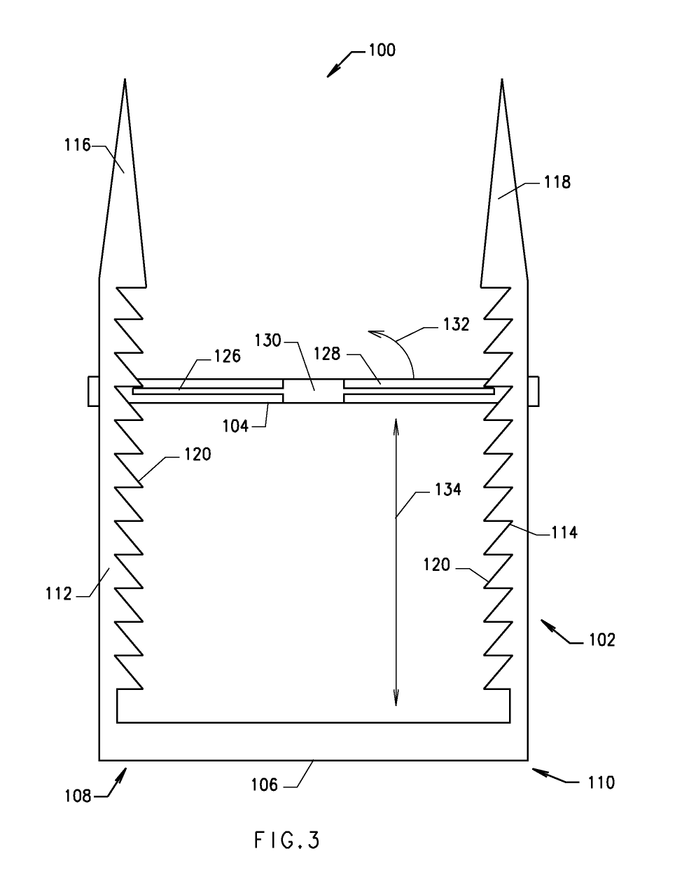

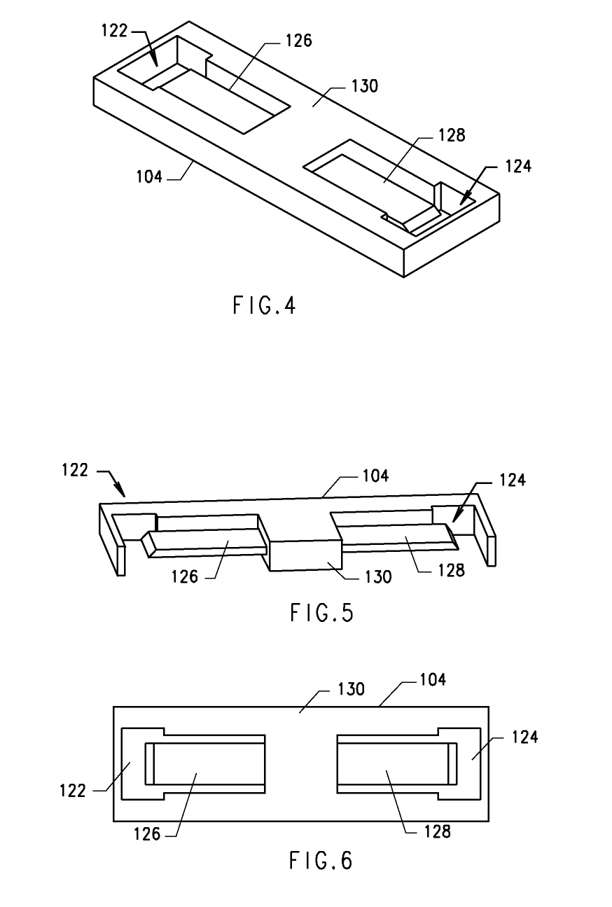

Referring to FIGS. 1-6, an embodiment of the ratcheting staple device is illustrated and generally indicated as 100. The ratcheting staple device 100 includes a staple body 102 that is used to puncture the tissue and a footplate 104 to mechanically engage the staple body 102. The footplate 104 secures the tissue between the staple body 102 and the footplate 104.

The staple body 102 includes a base 106 having a first end 108 and second end 110. Extending from the first end 108 and the second end 110 are a first leg 112 and a second leg 114, respectively. In one embodiment, the first leg 112 and the second leg 114 extend substantially perpendicular to the base 106, and terminate in tapered points 116 and 118. The first and second legs 108 and 110, respectively, may have a length between approximately 0.5 mm and approximately 10 mm. Preferably, the first leg 112 and the second leg 114 are the same length.

The first leg 112 includes a modified inner surface 120 that is configured to mechanically engage with the foot plate 104. For example, the modified inner surface 120 may include ratchet teeth, barbs, grooves, channels, or other recesses. In one aspect, the modified inner surface 120 protrudes from the first leg 112 and is angled toward the base 106. In another aspect, the modified inner surface 120 may be recessed into the body of the first leg 112. Although, not shown, the second leg 114 may also include a modified inner surface 120, similar to the modified inner surface 120 of the first leg 112.

In one aspect, the footplate 104 defines a first and second cavity 122 and 124 configured to receive the first leg 112 and the second leg 114, respectively. The footplate 104 also includes first and second locking members 126 and 128 to mechanically engage the respective modified inner surface 120 for each of the first leg 112 and the second leg 114. For example, the first and second locking members 126 and 128 may be semi-rigid pawls or unidirectional leaves that are cantilevered from a central portion 130 of the footplate 104, as shown in FIG. 2. The first and second locking members 126 and 128 slidably engage the modified inner surface 120 and pivot away, as indicated by 132 from the base 106 as the footplate 104 travels towards the base 106. The first and second locking members 126 and 128 cannot pivot towards the base 106, and therefore the footplate 104 is prevented from traveling in a retrograde motion once mechanically engaged to the modified inner surface 120. The distance between the base 106 footplate 104, indicated generally as 134, can be selectively controlled such that the secured tissue thickness can range between 0.6 mm to 9.0 mm.

In another aspect, the first and second locking members 126 and 128 may be protrusions that engagingly fit within recesses of the modified inner surface 120. In this aspect, the recesses reduce the potential for injury to the tissue being secured.

In yet another aspect, the footplate 104 may not define the first and second cavities 122 and 124. In this aspect, the first and second legs 112 and 114 are configured to puncture the surface of the footplate 104, thereby compressively engaging the first and second legs 112 and 114 to prevent retrograde motion along the modified inner surface 120.

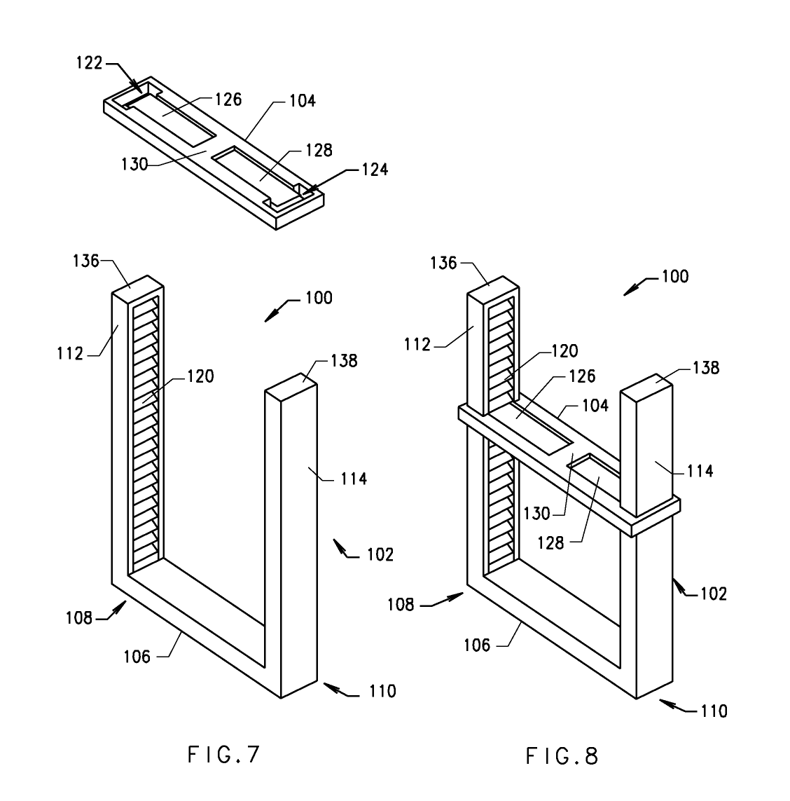

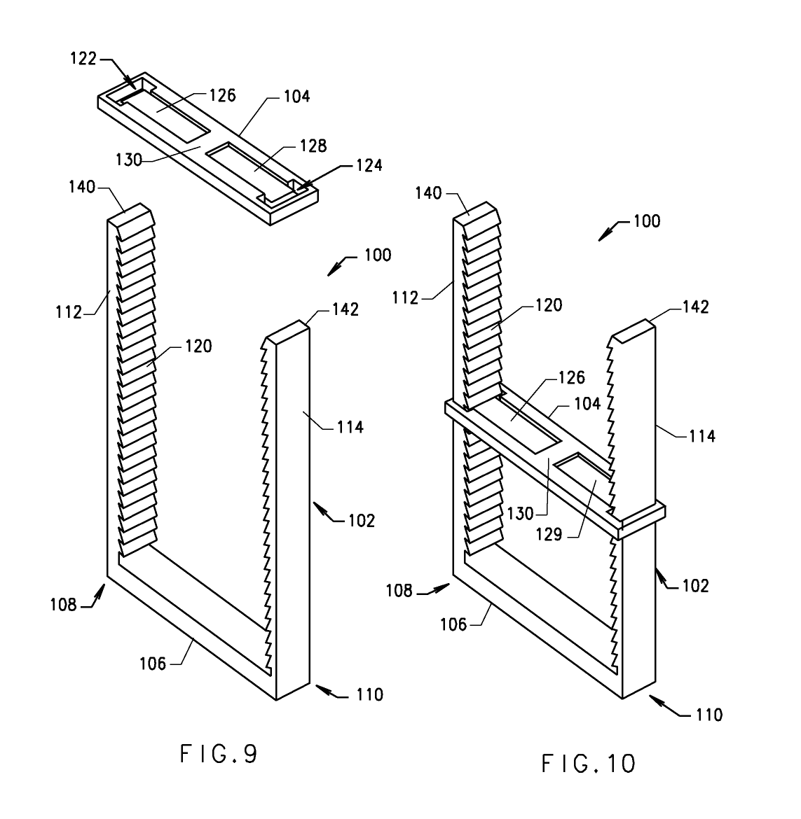

Referring to FIGS. 7 and 8, in some embodiments the ratcheting staple device 100 may have the first leg 112 and the second leg 114 both terminate in flat ends 136 and 138. In this aspect, the modified inner surfaces 120 are recessed within the first leg 112 and second leg 114, respectively. Similarly, in FIGS. 9 and 10, some embodiments of the ratcheting staple device 100 may have the first leg 112 and the second leg 114 that terminate in chisel-point ends 140 and 142.

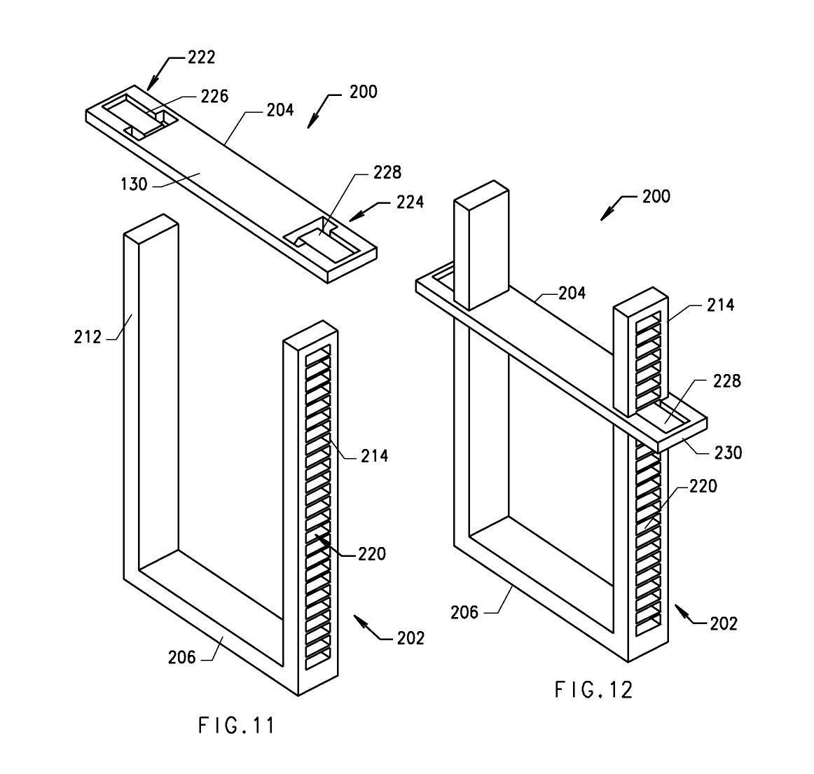

In one embodiment, shown in FIGS. 11-12, the ratcheting staple device, designated 200, may have a staple body 202 having a base 206, a first leg 212, and a second leg 214 with the first leg 212 and second leg 214 defining a modified outer surface 220. In this aspect, the footplate 204 defines a first and second cavity 222 and 224 configured to receive the first leg 212 and the second leg 214, respectively. The footplate 204 also includes first and second locking members 226 and 228 to mechanically engage the modified outer surfaces 220 for each of the first leg 212 and the second leg 214. For example, the first and second locking members 226 and 228 may be semi-rigid pawls or unidirectional leaves that are cantilevered from a peripheral edge 230 of the footplate 204. The first and second locking members 226 and 228 slidably engage the modified outer surfaces 220 and pivot away from the base 206 as the footplate 204 travels towards the base 206.

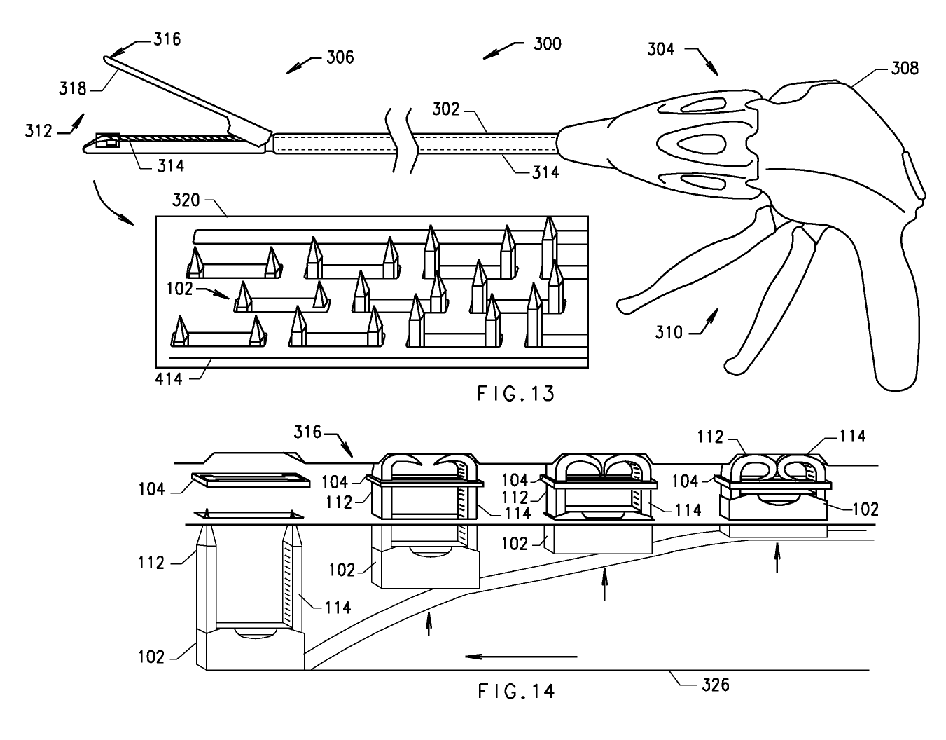

Referring to FIGS. 13 and 14, an embodiment of a stapler device is illustrated and generally indicated as 300. The stapler device 300 has an elongated body 302 having a proximal end 304 and a distal end 306. The proximal end of the elongated body 302 is attached to a handle 308 having a pivotable mounted trigger 310. The trigger 310 is in mechanical communication with a staple forming mechanism 312 attached to the distal end 306 of the elongated body 302.

In one aspect, the trigger 310 is in mechanical communication with the staple forming mechanism 312 through a lumen 314 defined by the elongated body 302. For example, the trigger 310 may be mechanically engaged with the staple forming mechanism 312 through an arrangement of wires, springs, cams, or other components (not shown) such that actuation of the trigger effectuates actuation of the staple forming mechanism 312.

The staple forming mechanism 312 includes a staple cartridge 314 axially aligned with the elongated body 302 and an anvil 316 having a footplate cartridge 318. As shown in the detailed view 320, the staple cartridge 314 includes one or more staple bodies 102. The anvil 316 is pivotably attached to the elongated body 302 and is configured to maintain a normal displacement 322 away from the staple cartridge 314. The anvil 316 rotates about a pivot 324 in response to actuation of the trigger 310.

As shown in FIG. 14, the actuation of the trigger 310 also causes a beveled ejector 326 for the staple cartridge 314 to engage the staple body 102 and eject the staple body 102 out of the staple cartridge 314. The staple body 102 is ejected perpendicular to the staple cartridge 314 to engage tissue (not shown), the footplate 104, and the anvil 316. In one embodiment, the footplate 104 includes a groove or recess (not shown) on the surface of the footplate 104 facing the anvil 316 to receive the legs 112 and 114 of the staple body 102 as the legs 112 and 114 are curved towards the footplate 104 by the anvil 316.

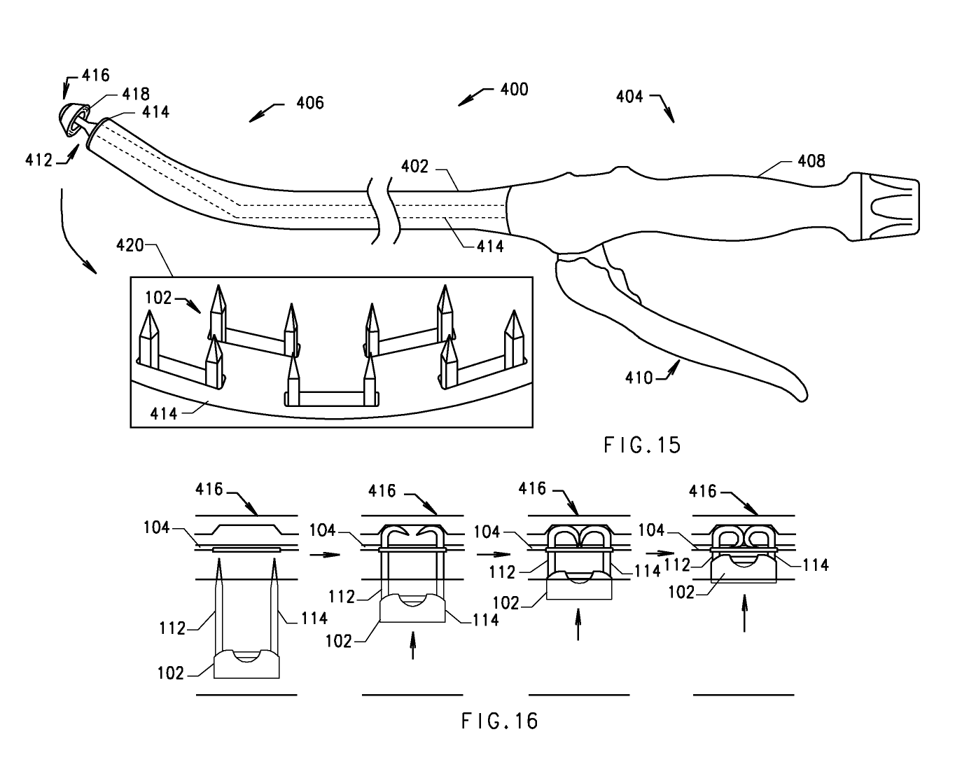

Referring now to FIGS. 15 and 16, an embodiment of a stapler device is illustrated and generally indicated as 400. The stapler device 400 has an elongated body 402 having a proximal end 404 and a distal end 406. The proximal end of the elongated body 402 is attached to a handle 408 having a pivotable mounted trigger 410. The trigger 410 is in mechanical communication with a staple forming mechanism 412 attached to the distal end 406 of the elongated body 402.

In one aspect, the trigger 410 is in mechanical communication with the staple forming mechanism 412 through a lumen 414 defined by the elongated body 402. For example, the trigger 410 may be mechanically engaged with the staple forming mechanism 412 through an arrangement of wires, springs, cams, or other components (not shown) such that actuation of the trigger effectuates actuation of the staple forming mechanism 412.

The staple forming mechanism 412 includes a circular staple cartridge 414 attached to the distal end 406 of the elongated body 402 and perpendicular to a central axis 700 of the elongated body 402. As shown in the detailed view 420, the staple cartridge 414 includes one or more of the staple body 102. The staple forming mechanism 412 also includes an anvil 416 having a footplate cartridge 418. The anvil 416 is attached to the elongated body 402 through a piston 424 and is configured to be parallel to and facing the staple cartridge 414. The anvil 416 is brought into engagement with the staple cartridge 414 upon compression of the piston 424 in response to actuation of the trigger 410.

As shown in FIG. 14, the actuation of the trigger 410 also causes an ejector (not shown) of the staple cartridge 414 to engage the staple body 102 and eject the staple body 102 out of the staple cartridge 414. The staple body 102 may be ejected perpendicular to the staple cartridge 414 to engage tissue (not shown), the footplate 104, and the anvil 416.

It is believed that the present disclosure and many of its attendant advantages will be understood by the foregoing description, and it will be apparent that various changes may be made in the form, construction, and arrangement of the components without departing from the disclosed subject matter or without sacrificing all of its material advantages. The form described is merely explanatory, and it is the intention of the following claims to encompass and include such changes.

While the present disclosure has been described with reference to various embodiments, it will be understood that these embodiments are illustrative and that the scope of the disclosure is not limited to them. Many variations, modifications, additions, and improvements are possible. More generally, embodiments in accordance with the present disclosure have been described in the context of particular implementations. Functionality may be separated or combined in blocks differently in various embodiments of the disclosure or described with different terminology. These and other variations, modifications, additions, and improvements may fall within the scope of the disclosure as defined in the claims that follow.

Those skilled in the art will appreciate that variations from the specific embodiments disclosed above are contemplated by the invention. The following invention should not be restricted to the above embodiments, but should be measured by the following claims.

What is claimed is:

1. A surgical staple device comprising:
a staple body defining a first leg, a second leg, and a base having a first end and a second end, the first leg extending from the first end and the second leg extending from the second end, the first leg having a first inner surface and a first outer surface and the second leg having a second inner surface and a second outer surface, wherein the first inner surface and the second inner surface face have modified surfaces to prevent retrograde motion of the surgical staple device; and
a footplate mechanically engaged the modified surfaces, the footplate having a first footplate end defining a first channel to receive the first leg and a second footplate end defining a second channel to receive the second leg.

2. The device of claim 1, wherein the footplate further comprises a locking mechanism to securely engage the modified surfaces.

3. The device of claim 2, wherein the locking mechanism comprises:
at least one unidirectional leaf-pawl to slidably engage the modified surfaces, the at least one leaf-pawl pivotally mounted to the footplate, wherein the at least one-leaf pawl pivots away from the base as the footplate is advanced towards the base.

4. The device of claim 1, wherein the modified surface includes at least one of a plurality of ratchet teeth, a plurality of barbs, a plurality of grooves, and a plurality of corrugated channels.

5. The device of claim 1, wherein the modified surface includes a plurality of recesses.

6. The device of claim 1, wherein the first leg and the second leg have a quadrilateral cross-section.

7. The device of claim 1, wherein the first leg and the second leg have a triangular cross-section.

8. The device of claim 1, wherein the first leg and the second leg have a hemispherical cross-section.

9. The device of claim 1, wherein the first leg and the second leg have a tapered configuration.

10. The device of claim 1, wherein the first leg and the second leg terminate at chisel-points.

11. The device of claim 1, wherein the first leg and the second leg terminate in flat ends.

12. A surgical staple device comprising:
a staple body defining a first leg, a second leg, and a base having a first end and a second end, the base having a first leg extending from the first end and a second leg extending from the second end, the first leg having a first outer surface and a first outer surface and the second leg having a second outer surface and a second outer surface, wherein the first outer surface and the second outer surface face have modified surfaces to prevent retrograde motion of the surgical staple device; and
a footplate mechanically engaged the modified surfaces, the footplate having a first footplate end defining a first channel to receive the first leg and a second footplate end defining a second channel to receive the second leg.

13. The device of claim 12 wherein the footplate further comprises a locking mechanism to securely engage the modified surfaces.

14. The device of claim 13, wherein the locking mechanism comprises:
at least one unidirectional leaf-pawl to slidably engage the modified surfaces, the at least one leaf-pawl pivotally mounted to the footplate, wherein the at least one-leaf pawl pivots away from the base as the footplate is advanced towards the base.

15. The device of claim 12 wherein the modified surface includes at least one of a plurality of ratchet teeth, a plurality of barbs, a plurality of grooves, and a plurality of corrugated channels.

16. The device of claim 12, wherein the modified surface includes a plurality of recesses.

17. A surgical staple device comprising:
a staple body defining a first leg, a second leg, and a base having a first end and a second end, the base having a first leg extending from the first end and a second leg extending from the second end, the first leg having a first surface and the second leg having a second surface, wherein the first surface and the second surface are modified surfaces to prevent retrograde motion of the surgical staple device; and
a footplate mechanically engaged the modified surfaces, the footplate having a first footplate end defining a first channel to receive the first leg and a second footplate end defining a second channel to receive the second leg.

18. A surgical stapler device comprising:
an elongated shaft having a proximal end and a distal end, a handle mounted on the proximal end of the elongated shaft and a staple device forming mechanism mounted on the distal end of the elongated shaft, wherein the staple device forming mechanism includes a plurality of staple devices comprising:
a staple body defining a first leg, a second leg, and a base having a first end and a second end, the base having a first leg extending from the first end and a second leg extending from the second end, the first leg having a first surface and the second leg having a second surface, wherein the first surface and the second surface are modified surfaces to prevent retrograde motion of the staple body, and wherein the staple device forming mechanism further includes a footplate mechanism containing a plurality of footplates, each of the plurality of footplates configured to engage the modified surfaces of one of the plurality of staple devices, each of the plurality of footplates having a first footplate end defining a first channel to receive the first leg and a second footplate end defining a second channel to receive the second leg.

19. A method for manufacturing a surgical staple device comprising:
modifying a first surface at a first end of an elongated material to form a first modified surface having at least one of a first plurality of projections or a first plurality of recesses;
modifying a second surface at a second end of the elongated material to form a second modified surface having at least one of a second plurality of projections or a second plurality of recesses;
forming an open-ended staple body from the elongated material by bending the first end to form a first leg and bending the second end to form a second leg; and
providing a footplate defining a first channel at proximal end of the footplate and defining a second channel at a distal end of the footplate, wherein the first channel further defines a first pawl and the second channel further defines a second pawl;
wherein the first channel is configured to slidably engage the first leg and the first pawl is configured to mechanically engage the first modified surface; and
wherein the second channel is configured to slidably engage the second leg and the second pawl is configured to mechanically engage the second modified surface.

20. A method for using a surgical staple device comprising:
providing a surgical stapler device including an elongated shaft having a proximal end and a distal end, a handle mounted on the proximal end of the elongated shaft, a staple device forming mechanism mounted on the distal end of the elongated shaft, and a footplate mechanism mounted on the distal end of the elongated shaft opposite the staple device forming mechanism, the staple device forming mechanism further including a plurality of staple devices having modified surfaces and, the footplate mechanism including a plurality of footplates configured to mechanically engage the modified surfaces;
positioning a first portion of tissue adjacent to a second portion of tissue;
positioning the distal end of the elongated shaft to enclose the first portion of tissue and the second portion of tissue between the staple device forming mechanism and the footplate mechanism;
manipulating the handle to compress the first portion of tissue and the second portion of tissue between the staple device forming mechanism and the footplate mechanism;
wherein at least one of the plurality of staple devices pierces the first portion of tissue and the second portion of tissue; and
wherein at least one of the plurality of footplates mechanically engages the at least one of the plurality of staple devices compressing the first portion of tissue and the second portion of tissue there between.
